(19) United States
(12) Patent Application Publication
(10) Pub. No.: US 2012/0087466 A1
(43) Pub. Date: Apr. 12, 2012

(54) X-ray Image Recording Method

(75) Inventor: Klaus KLINGENBECK, Aufsess (DE)

(73) Assignee: SIEMENS AKTIENGESELLSCHAFT, Munich (DE)

(21) Appl. No.: 13/233,060

(22) Filed: Sep. 15, 2011

(30) Foreign Application Priority Data
Sep. 17, 2010 (DE) 10 2010 040 976.6

(51) Int. Cl. A61B 6/03; H05G 1/64

(52) U.S. Cl. 378/19; 378/98.12

(57) ABSTRACT

The method includes using an angiography recording system to record a series of projection images of the organ or hollow organ of an examination object during a contrast-agent wash-in or wash-out process; using a CT recording system to record and reconstruct a 3D volume image of the organ or hollow organ of the examination object; at least partly superposing the series of projection images or image sections from the series of projection images on the 3D volume image; and displaying the 3D volume image on which the projection images are superposed.

### PRIORITY STATEMENT

The present application hereby claims priority under 35 U.S.C. §119 on German patent application number DE 10 2010 040 976.6 filed Sep. 17, 2010, the entire contents of which are hereby incorporated herein by reference.

### FIELD

At least one embodiment of the invention generally relates to a method for recording a dynamic wash-in or wash-out process of a contrast agent in an organ or hollow organ of an examination object.

### BACKGROUND

X-ray diagnostic systems are standard equipment in medical imaging and are used, for example, for interventional therapy. Angiography systems, generally C-arm X-ray systems, are used for e.g. monitoring the therapy of vascular and cardiac disorders and for minimally invasive therapy in the case of tumors. As a result of their flat-panel X-ray detectors with pixel elements arranged in a matrix, they offer a very high spatial resolution (pixel size generally approximately 150 μm) and can be used for both 2D and 3D imaging (so-called DynaCT from Siemens). However, in the case of low-contrast resolution and in terms of the recording speed, conventional computed/tomography scanners are still superior in 3D imaging; on the other hand, the latter are disadvantageous in terms of resolution and field of view in 2D imaging.

In order to be able to use the advantages of both systems, DE 198 02 405 B4 for example discloses an X-ray diagnostic device, in which two recording systems are arranged on one rotatable gantry: they are a CT recording system with a line-shaped X-ray detector and an angiography recording system with a planar X-ray detector. The CT recording system can be used to implement the known CT modes, e.g. recording sequential slices with a so-called stop-and-shoot advance of a patient table, or spiral imaging with a continuous advance of the patient table and continuous gantry rotation. The angiography recording system can implement two known modes: 2D fluoroscopy with a stationary gantry and 3D rotational imaging (e.g. DynaCT) with a continuously or sequentially rotating gantry.

### SUMMARY

In at least one embodiment of the present invention, a method is provided by way of such an X-ray diagnostic device, which method ensures a particularly clear illustration of dynamic processes such as contrast agent wash-in or wash-out.

According to at least one embodiment of the invention, a method for recording a dynamic wash-in or wash-out process is disclosed. Advantageous embodiments of the invention respectively are the subject matter of the dependent claims.

The method according to at least one embodiment of the invention is for recording a dynamic wash-in or wash-out process of a contrast agent in an organ or hollow organ of an examination object using an X-ray diagnostic apparatus with a rotatable gantry, which X-ray diagnostic device has two recording systems arranged in the gantry, wherein a computed-tomography recording system has a first X-ray source and, arranged opposite to the first X-ray source, a computed-tomography X-ray detector with a row of individual detectors, and a second recording system, more particularly an angiography recording system, has a second X-ray source, which is arranged offset with respect to the first X-ray source, and, arranged opposite to the second X-ray source, a planar X-ray detector with pixel elements arranged in a matrix-like fashion. In at least one embodiment, the method comprises:
using the angiography recording system to record a series of projection images of the organ or hollow organ of the examination object during a contrast-agent wash-in or wash-out process,
using the CT recording system to record and reconstruct a 3D volume image of the organ or hollow organ of the examination object,
at least partly superposing the series of projection images or image sections from the series of projection images on the 3D volume image, and
displaying the 3D volume image on which the projection images are superposed.

### BRIEF DESCRIPTION OF THE DRAWINGS

The invention and further advantageous embodiments as per the features of the dependent claims are explained in more detail in the drawing below on the basis of schematically illustrated example embodiments, without this restricting the invention to these example embodiments. In the drawings:

FIG. 1 shows a known X-ray diagnostic device with two recording systems,

FIG. 2 shows a sequence of a method according to an embodiment of the invention, and

FIG. 3 shows a display of a 3D volume image with superposed color gradient.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

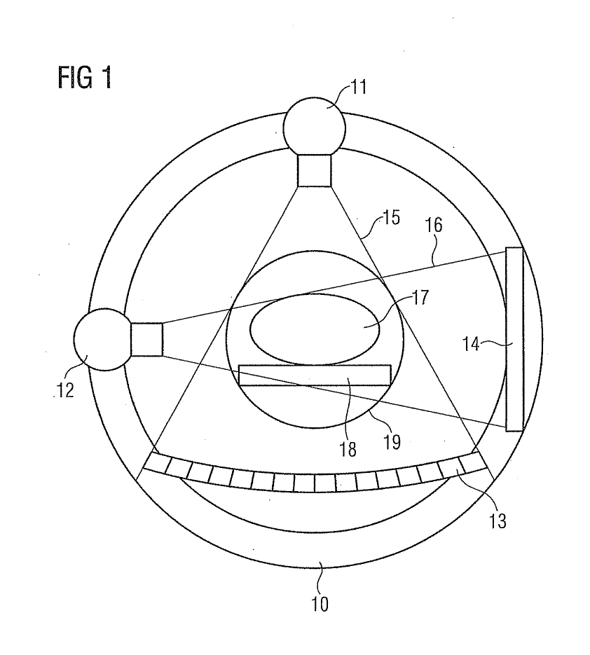

In a gantry 10, a known X-ray diagnostic apparatus, shown in FIG. 1, has a computed-tomography (CT) recording system with a first X-ray source 11 and a CT X-ray detector 13, and an angiography recording system with a second X-ray source 12 and a second, flat-panel X-ray detector 14. In the case of the CT recording system, the first X-ray source 11 emits a fan beam 19 and the CT X-ray detector 13 is arced and assembled from a row of individual detectors (e.g. 512). In order to scan an examination object 17 arranged on a patient table 18, the CT recording system is rotated through 360° around the examination object 17 by way of the gantry 10; the recorded data record may be reconstructed to form a 3D volume image.

The angiography recording system has a second X-ray source 12 and a flat-panel X-ray detector 14; the second X-ray source emits a conical X-ray beam 16 to the flat-panel X-ray detector 14. There is an angular offset between the first bisector from the first X-ray source 11 on the CT X-ray detector 13 and the second bisector from the second X-ray source 12 on the flat-panel X-ray detector 14, which angular offset may be used to describe the offset between CT recording system and angiography recording system. The angular offset is preferably 90°. It is possible to use the angiography recording system to record (2D) projection images, both in the case of a stationary gantry and in the case of a rotating gantry. A series of projection images recorded in the case of a rotating gantry may also be reconstructed to form a 3D image. There may be simultaneous or alternating operation of the CT recording system and the angiography recording system, as described in e.g. DE 198 02 405 B4, the entire contents of which are hereby incorporated herein by reference. By way of example, provision is made for a system control for actuating the X-ray diagnostic apparatus, which system control actuates both the CT recording system and the angiography recording system. Such a system control may also be formed by a control PC.

A registration between a 3D volume image from the CT recording system and a projection image from the angiography recording system, which registration is required for a superposition of the 3D volume image and the projection images, can for example be brought about as follows: The projection direction of a 2D projection image, recorded by way of the angiography recording system, with respect to the 3D volume image is established taking into account the geometric arrangement between CT recording system and angiography recording system. Since the geometric arrangement on the gantry between CT recording system and angiography recording system is fixed and known (angular offset), the projection direction of the angiography recording system relative to the CT recording system can be derived in a simple fashion and it is thus possible to establish the projection direction in the 3D volume image. A 2D projection image for the established projection direction of the angiography recording system is subsequently simulated, e.g. by way of a computational unit, from the 3D volume image or from the data record from which the 3D volume image was reconstructed. Such 2D projection images simulated from CT data records are known and are also referred to as digitally reconstructed radiographs (DRR).

Then the 2D projection image, recorded by way of the angiography recording unit, and the 2D projection unit, simulated for the same projection direction from the 3D data record, are matched to one another by e.g. taking magnification factors into account. The 3D volume image is then superposed on the recorded 2D projection image using the simulated 2D projection image; if need be the superposed images can be displayed on e.g. a display unit of the X-ray diagnostic apparatus.

The method according to an embodiment of the invention is particularly suitable for displaying dynamic processes such as a contrast-agent wash-in in a hollow organ or organ, and thereby simplifying the diagnosis for a user.

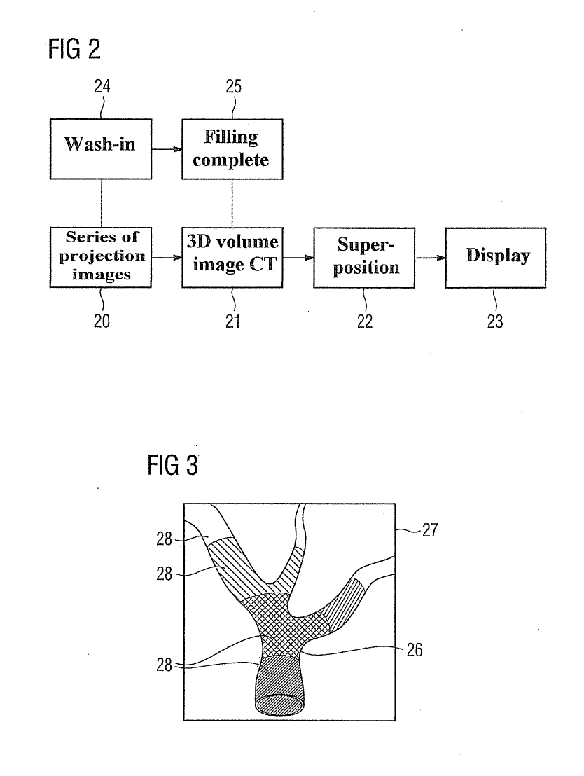

FIG. 2 shows the steps of the method according to an embodiment of the invention. The entire method can for example be actuated by the system control of the X-ray diagnostic apparatus; an image system may be used for image processing and display. In a first step 20, a series of projection images of an organ or hollow organ of an examination object are recorded by way of the angiography recording system. The recordings are recorded precisely during the time interval in which there is a wash-in (24) in the hollow organ or organ of a contrast agent that was previously administered to the examination object arterially or intravenously. As a result, the series of projection images documents the wash-in of the contrast agent in the hollow organ or organ.

The series of projection images may be recorded either in the case where the gantry is stationary, i.e. in the case of a single recording position of the angiography recording system, or in the case where the gantry rotates around the examination object, i.e. at different recording positions (projection angles). The series of projection images may be processed; by way of example, noise corrections may be carried out or a mask image may be subtracted to image merely the flow of the contrast agent through the hollow organ. In addition to a wash-in, it is also possible to record a wash-out of a contrast agent in the hollow organ or organ.

A 3D volume image is generated by way of the CT recording system using a method known per se in a second step 21, more particularly from a series of 2D CT images that are reconstructed to form the 3D volume image. By way of example, these recordings are recorded precisely during the filled phase 25, i.e. during the time interval in which the hollow organ or organ is completely filled by the contrast agent. The second step 21 may also take place after the wash-out of the contrast agent, or it may precede the first step 20, that is to say e.g. before the contrast-agent injection. In the case of a wash-in and a CT recording subsequently carried out during the filled phase, the wash-in detected by way of the angiography recording system may also be used as a trigger for starting the CT recording system (angiographic bolus tracking).

The series of projection images (in processed or unprocessed form) or at least part of the series or sections of the series are superposed on the reconstructed 3D volume image in a third step 22 and the 3D volume image is displayed in a fourth step 23. Here the superposition may also be carried out before or at the same time as the display, i.e. online. By way of example, the 3D volume image is displayed as maximum intensity projection (MIP) or volume rendering technique (VRT), wherein the recorded hollow organ or organ is displayed on the display unit in a rotatable fashion in order to generate image views from different sides and from different angles. These angles can be correlated with the projection images at the corresponding recording positions (projection angles) of the angiography recording system. The corresponding projection images or processed projection images can then respectively be displayed superimposed on the 3D volume image for the current image view of the 3D volume image. Thus, when the 3D volume image is rotated on the display device, the respectively superimposed projection images change in accordance with the current image view.

In addition to this, the temporal and spatial progress of the contrast agent is illustrated by way of the projection images in color gradients. By way of example, this is shown in FIG. 3 on the basis of a hollow organ 26 on the display 27 as a 3D volume image with a superimposed color gradient 28. By way of example, this can be implemented by virtue of the fact that a mask image is in each case subtracted from the projection images in order to image merely the flow of the contrast agent through the hollow organ, and by coloring the projection images processed thus and superposing them on the 3D volume image. By way of example, the color gradient progressing from neutral yellow tones to a neutral red tone by increasingly adding red tones or the like can be used as color gradient.

Even if the projection images were recorded whilst the gantry was rotating, and hence there are a multiplicity of projection images available that were recorded at different recording positions, it may nevertheless occur that for the currently displayed image view of the 3D volume image there is no projection image that was recorded at the corresponding recording position. In such a case, one or more interpolated projection images may be simulated, with the projection images respectively recorded at adjacent recording positions being used for the interpolation. The simulated projection images are likewise superposed like the recorded projection images and displayed in a superimposed fashion in the case of the corresponding image view of the 3D volume image. The color gradients for displaying the temporal and spatial progress of the contrast agent can likewise be simulated and superimposed.

In the case where the projection images were recorded by way of the angiography recording system when the gantry was stationary, i.e. if they were recorded at only a single recording position, the correlation between static CT anatomy and dynamic angiography is only implemented at one fixed observation angle.

An end result of the method according to an embodiment of the invention is the creation of a spatial 3D volume image of the hollow organ or organ, which may be observed in different image views and at different angles, and in which the flow is registered in a color-coded fashion for each angle.

An embodiment of the invention may briefly be summarized as follows: For the purposes of improved diagnosis, provision is made for a method for recording a dynamic wash-in or wash-out process of a contrast agent in an organ or hollow organ of an examination object using an X-ray diagnostic apparatus with a rotatable gantry, which X-ray diagnostic device has two recording systems arranged in the gantry, wherein a computed-tomography recording system has a first X-ray source and, arranged opposite to the first X-ray source, a computed-tomography X-ray detector with a row of individual detectors, and a second recording system, more particularly an angiography recording system, has a second X-ray source, which is arranged offset with respect to the first X-ray source, and, arranged opposite to the second X-ray source, a planar X-ray detector with pixel elements arranged in a matrix-like fashion, the method comprising:
using the angiography recording system to record a series of projection images of the organ or hollow organ of the examination object during a contrast-agent wash-in or wash-out process,
using the CT recording system to record and reconstruct a 3D volume image of the organ or hollow organ of the examination object,
at least partly superposing the series of projection images or image sections from the series of projection images on the 3D volume image, and
displaying the 3D volume image on which the projection images are superposed.

The method according to at least one embodiment of the invention affords the possibility of ensuring a particularly accurate, easily identifiable, and for this reason easily diagnosable display of the dynamic process of the wash-in or wash-out of a contrast agent in hollow organs or organs of an examination object. By superposing 2D projection images of the dynamic process on a 3D volume image of a static state of the examination object (e.g. complete filling), it is possible to visualize, in a simple fashion and with high image quality, both the temporal and spatial progress of the contrast agent in the precise anatomy of the examination object.

Thus, the method according to at least one embodiment of the invention supports a particularly accurate diagnosis of e.g. perfusion disorders in hollow organs/organs and thereby substantially simplifies the interpretation of abnormal changes of organs or hollow organs of the examination object. The fixed geometric arrangement of the two recording systems of the X-ray diagnostic apparatus with respect to one another, and hence the simple and error-free assignment between 3D volume image and 2D projection images, is utilized for simplification during the display.

The 3D volume image is advantageously recorded before or after the wash-in or wash-out process, more particularly during a static state of the examination object with respect to a contrast agent. An example of this is the phase in which the organ or hollow organ is completely filled with the contrast agent; another example is a phase before contrast agent is supplied to the examination object.

According to one alternative of at least one embodiment of the invention, the series of projection images is recorded during a rotation of the gantry and the projection images are recorded at different recording positions of the recording system with respect to the examination object. In another alternative, the series of projection images is recorded when the gantry is stationary, i.e. at a single recording position of the recording system with respect to the examination object.

According to one embodiment of the invention, the superposition is performed such that projection images or image sections from the projection images recorded at different times are displayed in different colors. More particularly, the progress of the contrast agent in the hollow organ or organ is displayed by way of a color gradient that is superimposed into the 3D volume image.

According to a further embodiment of the invention, the superposed 3D volume image is displayed in a rotatable fashion on a display device, i.e. such that the 3D volume image can be observed from all sides. In the process, the display can be rotated manually or automatically in front of the eyes of an observer.

According to a further embodiment of the invention, the respectively displayed image view of the rotatable 3D volume image is such that the projection images or image sections from the projection images that respectively correspond to the shown image view are respectively superimposed. Here, the projection images that correspond to the image view are understood to mean the projection images from a recording position that are projected into the same plane, which plane is the current observation plane on the display. Thus, for example, provision can be made for the projection images not to be superimposed in this image view, which projection images were recorded at different recording positions.

If there is no projection image available that corresponds to the image view, a projection image that is interpolated between two projection images of adjacent image views is determined (that is to say e.g. calculated), superposed on the 3D X-ray image, and superimposed on the display.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for recording a dynamic wash-in or wash-out process of a contrast agent in an organ or hollow organ of an examination object using an X-ray diagnostic apparatus with a rotatable gantry, the X-ray diagnostic apparatus including two recording systems arranged in the gantry, wherein a first recording system includes a first X-ray source and, arranged opposite to the first X-ray source, an X-ray detector with a row of individual detectors, and wherein a second recording system includes a second X-ray source, arranged offset with respect to the first X-ray source, and, arranged opposite to the second X-ray source, a planar X-ray detector with pixel elements arranged in a matrix-like fashion, the method comprising:
recording, using the second recording system, a series of projection images of the organ or hollow organ of the examination object during a contrast-agent wash-in or wash-out process;
recording and reconstructing, using the first recording system, a 3D volume image of the organ or hollow organ of the examination object;
at least partly superposing the series of projection images or image sections from the series of projection images on the 3D volume image; and
displaying the 3D volume image on which the series of projection images or image sections from the series of projection images are superposed.

2. The method as claimed in claim 1, wherein the 3D volume image is recorded before or after the wash-in or wash-out process.

3. The method as claimed in claim 1, wherein the series of projection images is recorded during a rotation of the gantry and the wherein the projection images are recorded at different recording positions.

4. The method as claimed in claim 1, wherein the series of projection images is recorded when the gantry is stationary.

5. The method as claimed in claim 1, wherein the superposition is performed such that projection images or image sections from the projection images recorded at different times are displayed in different colors.

6. The method as claimed in claim 1, wherein the superposed 3D volume image is displayed in a rotatable fashion.

7. The method as claimed in claim 3, wherein the respectively displayed image view of the rotatable 3D volume image is such that the projection images or image sections from the projection images that correspond to the image view are superimposed.

8. The method as claimed in claim 7, wherein, if there is no projection image available that corresponds to the image view, a projection image that is interpolated between two projection images of adjacent image views is determined and superimposed.

9. The method as claimed in claim 1, wherein the displaying includes interactively displaying.

10. The method as claimed in claim 1, wherein the first recording system is a computed-tomography recording system and wherein the second recording system is an angiographic recording system.

11. A tangible computer readable medium including program segments for, when executed on a computer device, causing the computer device to implement the method of claim 1.

12. The method as claimed in claim 1, wherein the 3D volume image is recorded before the wash-in or wash-out process.

13. The method as claimed in claim 1, wherein the 3D volume image is recorded after the wash-in or wash-out process.

14. The method as claimed in claim 5, wherein the respectively displayed image view of the rotatable 3D volume image is such that the projection images or image sections from the projection images that correspond to the image view are superimposed.

15. The method as claimed in claim 6, wherein the respectively displayed image view of the rotatable 3D volume image is such that the projection images or image sections from the projection images that correspond to the image view are superimposed.

16. A method, comprising:
recording, using an angiographic recording system, a series of projection images of an organ or hollow organ of an examination object during a contrast-agent wash-in or wash-out process;
recording and reconstructing, using a computed tomography recording system, a 3D volume image of the organ or hollow organ of the examination object, the angiographic and computed tomography systems being arranged together in a gantry;
at least partly superposing the series of projection images or image sections from the series of projection images on the 3D volume image; and
displaying the 3D volume image on which the series of projection images or image sections from the series of projection images are superposed.

17. The method as claimed in claim 16, wherein the 3D volume image is recorded before the wash-in or wash-out process.

18. The method as claimed in claim 16, wherein the 3D volume image is recorded after the wash-in or wash-out process.

19. A tangible computer readable medium including program segments for, when executed on a computer device, causing the computer device to implement the method of claim 16.
